(19) United States
(12) Patent Application Publication
(10) Pub. No.: US 2012/0101077 A1
(43) Pub. Date: Apr. 26, 2012

(54) AGGLOMERATE FORMULATIONS USEFUL IN DRY POWDER INHALERS

(75) Inventors: Preetanshu Pandy, Springfield, NJ (US); Sai Prasanth Chamarthy, Springfield, NJ (US); Brent Ashley Donovan, Berkeley Heights, NJ (US)

(73) Assignee: Schering Corporation, Kenilworth, NJ (US)

(21) Appl. No.: 13/266,070

(22) PCT Filed: Apr. 23, 2010
(86) PCT No.: PCT/US10/32220

§ 371 Date: Jan. 9, 2012

Related U.S. Application Data
(60) Provisional application No. 61/172,343, filed on Apr. 24, 2009.

(51) Int. Cl. A61K 31/58; A61P 11/00

(52) U.S. Cl. 514/172

(57) ABSTRACT

Several embodiments of the present invention provide for an agglomerate useful for an agglomerate based dry powder inhaler comprising at least one active pharmaceutical agent, at least one additional functional excipient and at least one excipient, such as a binder. Useful at least one additional functional excipients include but are not limited to magnesium stearate, colloidal silica, silicon dioxide, sucrose stearate, L-leucine and combinations thereof.

### FIELD OF THE INVENTION

Various embodiments of the present invention relate to dry powder inhalers and, more particularly, to agglomerates that yield a desirable fine particle fraction.

### BACKGROUND

Drug delivery to the lungs can be accomplished with dry powder inhalers (DPIs), metered dose inhalers, and nebulizers. The majority of DPIs are passive, meaning they are ‘breath-actuated’ devices where the patient provides the energy to aerosolize the powder during the inhalation. In order to deposit drug in the respiratory tract, DPIs deliver micron-sized drug particles having an aerodynamic diameter of approximately 1-5 μm. Particles of this size have a high surface area and a large number of contact points between particles. The dominant interparticle interactions for such systems are Van der Waals and Columbic interactions. DPI formulations have proved challenging since micronized powders tend to be cohesive and flow poorly, both of which result in poor aerosolization efficiency and delivery of the drug.

Common types of DPIs include an inhaler with a micronized powder in a packet or capsule, a carrier formulation based DPI or an agglomerate formulation based DPI. In the carrier-based system, micronized drug is mixed with a coarse excipient, typically between 60 and 90 microns. α-Lactose monohydrate is the most widely used carrier, although alternative carriers, such as sorbitol, xylitol and mannitol, have been studied. In a carrier-based system, the micronized drug adheres to the larger carrier particle. When the particles are entrained in the airstream during an inhalation, the drug separates from the surface of the carrier and is inhaled while the larger carrier particle impacts in the oropharynx and is cleared.

Another formulation approach is the agglomerate-based system. In this technique, micronized drug may be agglomerated with an excipient as used in PULMICORT TURBOHALER® dry powder inhaler (AstraZeneca, Wilmington, Del.) Alternatively, micronized drug may be combined with micronized excipient as used in ASMANEX TWISTHALER® dry powder inhaler (Schering-Plough, Kenilworth, N.J.) and are formulated into agglomerates as described in U.S. Pat. No. 6,503,537, which is incorporated herein in its entirety. During the patient's inhalation, turbulence and collisions between agglomerates and the inhaler walls break these agglomerates into fine drug and excipient particles.

A major difference between a carrier-based formulation and agglomerate-based formulation is that for the agglomerate-based formulation, the micronized drug as well as the micronized excipient gets inhaled into the deep lung, whereas, in carrier based systems, the large carrier particles do not reach the lung because they generally get stuck in the throat and other areas of the body before the lung. Thus, agglomerate-based systems have unique challenges since most of the powder from the agglomerate is inhaled into the lung. Generally, it is desirable to inhale the least amount of powder into the lung. Thus, it would be desirable to increase the efficiency of agglomerate based formulations by increasing the desirable fine particles (fine particle fraction or FPF) of the formulation that can reach the target areas of the lung to treat various respiratory diseases, such as asthma and chronic obstructive pulmonary disease (COPD) and to reduce the total amount of powder that needs to be inhaled from the DPI.

### SUMMARY

Agglomerate formulations and methods that are capable of controlling and increasing the fine particle fraction of agglomerate-based DPI systems were surprisingly discovered. Several embodiments of the present invention provide for an agglomerate formulation useful for an agglomerate based dry powder inhaler comprising at least one active pharmaceutical agent, at least one binder and at least one additional functional excipient capable of changing the fine particle fraction of the delivered dose of the agglomerate, called hereinafter at least one additional functional excipient. The concentration of the at least one additional functional excipient may affect the magnitude of change of the fine particle fraction or fine particle dose. Thus, the performance of the various embodiments of the present invention may depend on the type of additive and the concentration of the additive.

Various embodiments of the present invention provide for an agglomerate useful for an agglomerate based dry powder inhaler comprising at least one active pharmaceutical agent, at least one binder and at least one additional functional excipient capable of changing the fine particle fraction of the delivered dose of the agglomerate. The at least one additional functional excipient may be selected from the group consisting sugars, lubricants, antistatic agents, amino acids, peptides, surfactants, phospholipids and combinations thereof. Specifically, the at least one additional functional excipient is selected from the group consisting of colloidal silica, magnesium stearate, sucrose stearate, lactose, glucose and mannitol, leucine and combinations thereof. More specifically, the at least one additional functional excipient is a lubricant and can be present in an amount from about 0.1 to about 10% of the total weight of the agglomerate, from about 0.5 to about 2% of the total weight of the agglomerate, about 1.0% of the total weight of the agglomerate or about 0.5% of the total weight of the agglomerate. The at least one binder is selected from the group consisting of lactose anhydrous NF, lactose monohydrate and combinations thereof or preferably, lactose anhydrous NF. When DPI is actuated, the active pharmaceutical agent emitted dose from a dry powder inhaler may have a fine particle fraction of greater than about 50% or greater than about 70%.

Alternative embodiments of the present invention provide for an agglomerate comprising at least one active pharmaceutical agent, lactose and magnesium stearate. Still other embodiments provide for an agglomerate comprising at least one active pharmaceutical agent, lactose and colloidal silica.

Further embodiments of the present invention provide a method of controlling the fine particle dose of an agglomerate particle based dry powder inhaler comprising an agglomerate formulation comprising at least one active pharmaceutical agent, at least one binder and at least one additional functional excipient capable of changing the fine particle fraction of the delivered dose of the agglomerate. The at least one additional functional excipient may be magnesium stearate and/or colloidal silica and can be present in an amount from about 0.1 to about 10% of the total weight of the agglomerate, about 1.0% of the total weight of the agglomerate or about 0.5% of the total weight of the agglomerate.

### BRIEF DESCRIPTION OF THE DRAWINGS

FIG. 1. SEM of a typical agglomerate-based formulation

FIG. 2a Effect of adding Magnesium stearate (MgSt) on the fine particle dose when MgSt is added at the final blending step of the process

FIG. 2b Effect of adding MgSt on the fine particle fraction when MgSt is added at the final blending step of the process

FIG. 3a Effect of adding MgSt on the fine particle fraction when MgSt is pre-blended with APA

FIG. 3b Effect of adding MgSt on the fine particle dose when MgSt is pre-blended with APA

FIG. 4a Effect of adding MgSt on the fine particle fraction when MgSt is pre-blended with the excipient (lactose in this case)

FIG. 4b Effect of adding MgSt on the fine particle dose when MgSt is pre-blended with the excipient (lactose in this case)

### DETAILED DESCRIPTION

The present invention surprisingly discovered agglomerate formulations and methods that are capable of controlling and increasing the fine particle fraction of agglomerate-based DPI systems. Several embodiments of the present invention provide for an agglomerate formulation useful for an agglomerate based dry powder inhaler comprising at least one active pharmaceutical agent, at least one binder and at least one at least one additional functional excipient. Other embodiments provide for an agglomerate formulation comprising an active pharmaceutical agent, magnesium stearate and lactose or an agglomerate formulation comprising an active pharmaceutical agent, colloidal silica and lactose. Still other embodiments provide for a method of controlling the fine particle dose of an agglomerate particle based dry powder inhaler comprising adding at least one additional functional excipient in the agglomerate formulation.

An agglomerate in accordance with the present invention is a bound mass of small particulates. Agglomerates may include at least one first material and at least one excipient, such as a solid binder. The first material, in accordance with the present invention can be anything as the present invention can be used broadly to make free-flowing agglomerates for any application including, medicine, cosmetics, food and flavoring, and the like. Desirably, the first material is an active pharmaceutical agent or drug which is to be administered to a patient in need of some course of treatment.

The at least one additional functional excipient does not appear to affect the agglomerate formation process at the concentration levels tested (0.5-2.0% w/w). Agglomerates with colloidal silica yielded agglomerates with higher fine particle fraction (FPF). Agglomerates with colloidal silica demonstrated an increase in fine particle fraction and fine particle dose. Agglomerates with magnesium stearate demonstrated an increase in fine particle dose. The concentration of the at least one additional functional excipients may affect the magnitude of change of the fine particle fraction or fine particle dose. The performance of the various embodiments of the present invention may depend on the type of additional functional excipient and the concentration of the additive.

Useful at least one additional functional excipients include but are not limited to sugars, lubricants, antistatic agents, amino acids, peptides, surfactants, phospholipids and combinations thereof. More specifically, Useful at least one additional functional excipients include but are not limited to colloidal silica, magnesium stearate, sucrose stearate, lactose, glucose and mannitol, leucine and combinations thereof. Useful magnesium stearate include but are not limited to the hydrates, such as monohydrate, dihydrate and trihydrate.

Magnesium stearate is a hydrophobic excipient commonly used in solid-dosage formulations to improve the flow of the bulk powder and to act as a lubricating aid to keep the powder from sticking and clogging the equipment. Several studies have investigated the use of magnesium stearate in dry powder carrier based formulations for inhalation. Addition of 0.5% w/w magnesium stearate in the presence of lactose fines resulted in an increase of particles in the respirable range than when lactose fines or magnesium stearate were used alone. This increase of respirable particles may be attributed to magnesium stearate reducing the electrostatic repulsion between lactose particles, so fine lactose increasingly attached to lactose carrier. Another study found that 0.5% w/w magnesium stearate reduced the fine particle fraction in a formulation of micronized particles, indicating some disparity in the literature concerning the effects of this additive, Westmeier, R. and Steckel, H., 2008. Combination particles containing salmeterol xinafoate and fluticasone propionate: formulation and aerodynamic assessment. J. Pharm. Sci., 97, 2299-2310. Thus, it is not clear whether magnesium stearate is always desirable to be included in carrier based DPI formulations. PULVINAL® Beclomethasone dipropionate (Trinity-Chiesi Pharmaceuticals, Cheshire, UK) is a DPI containing magnesium stearate that has already been approved in the European Union.

Colloidal silica (or untreated fumed silica) is an excipient used for many different applications in the pharmaceutical industry, though for carrier based dry powder systems, it may be used to promote free flow and absorb moisture on the surface of the powder (from Cabot Corporation product information sheets).

Additional functional excipients to DPI formulations have been studied in carrier based DPI systems, however, because of differences between carrier and agglomerate-based systems, the technologies for improving flow and aerosolization do not necessarily transfer from one system to the other. Specifically, additional excipients such as lubricants including magnesium stearate and colloidal silica have been used as anti-adherant agents in carrier based formulations useful in dry powder inhalers such as those described in WO2008000482. However, additional functional excipients have not been used in agglomerate formulation for agglomerate based dry powder inhalers. One reason that additional function excipients have been avoided in agglomerate based systems may be due to the lubricant properties of such excipients since, a priori, these properties would be considered to be undesirable to form an agglomerate particle. In particular, it may have been believed that adding a lubricant to an agglomerate formulation may undesirably weaken the agglomerate or prematurely deagglomerate the agglomerate if an lubricant excipient was included therein. Agglomerate formulations must be hard enough not to prematurely separate prior to actuation of the DPI. The agglomerate formulation must be hard enough to withstand forces during product shipping and handling while it is idling in the reservoir in the DPI as well as throughout the manufacturing process. Thus, it was surprisingly found that including at least one additional functional excipients to an agglomerate formulation can control and increase the fine particle fraction of the emitted dose of an agglomerate particle based dry powder inhaler and still provide agglomerate with an acceptable hardness.

Various embodiments of the present invention provided for agglomerate formulations that include at least one additional functional excipients and when emitted from a DPI result in an increase in the fine particle fraction of product delivered to the lung. Such agglomerates are useful in dry powder inhaler systems, such as the TWISTHALER®, sold by Schering-Plough.

Useful amounts of the at least one additional functional excipients include concentrations from about 0.1 to about 10.0% w/w, from about 0.1 to about 5.0% w/w, from about 0.5 to about 5.0% w/w, from about 0.5 to about 2.0% w/w, from about 0.5 to about 1.0% w/w, or about 0.5% or about 1%. In various embodiments of the present invention, the blending order of the at least one additional functional excipients were varied.

Suitable at least one additional functional excipient can be added during different stages of agglomerate manufacturing to achieve the desired effect on fine particle fraction of the agglomerate-based formulation. For example at least one additional functional excipients can be pre-blended with APA, and/or pre-blended with the excipients, and/or added during the last step of blending.

Useful excipients include binders which include but are not limited to lactose, such as lactose anhydrous NF, lactose monohydrate or combinations thereof.

Several other embodiments provide for a dosing system comprising a DPI and an agglomerate; wherein when the DPI is actuated and the agglomerate is delivered, an actuated dose comprises a fine particle fraction of at least 30%, at least 40%, at least 50%, at least 60% at least 70%, at least 75%, or at least 80%.

Agglomerates of APA or drug may be utilized and manufactured as described in U.S. Pat. No. 6,503,537, which is incorporated in its entirety herein. Any method of agglomerating the solid binder and the pharmacologically active agent may be used. Useful agglomerating methods include those which can be accomplished without converting the amorphous content of the solid binder to a crystalline form, prematurely, and which does not require the use of additional binder, can be practiced in accordance with the present invention.

An agglomerate in accordance with the present invention is a bound mass of small particulates. The agglomerates include at least one first material and at least one solid binder. The first material, in accordance with the present invention can be anything as, indeed, the present invention can be used broadly to make free-flowing agglomerates for any application including, medicine, cosmetics, food and flavoring, and the like. However, preferably, the first material is an active pharmaceutical agent or drug which is to be administered to a patient in need of some course of treatment.

The active pharmaceutical agent may be administered prophylactically as a preventative or during the course of a medical condition as a treatment or cure. The active pharmaceutical agent or drug may be a material capable of being administered in a dry powder form to the respiratory system, including the lungs. For example, a drug in accordance with the present invention could be administered so that it is absorbed into the blood stream through the lungs. More preferably, however, the active pharmaceutical agent is a powdered drug which is effective to treat some condition of the lungs or respiratory system directly and/or topically.

Useful agglomerates include agglomerates ranging in size from between about 100 to about 1500 μm. The agglomerates may have an average size of between about 300 and about 1,000 μm. Useful agglomerates may have a bulk density which ranges from between about 0.2 to about 0.4 g/cm³ or between about 0.29 to about 0.38 g/cm³.

It is useful to have a tight particle size distribution. In this context, particle size refers to the size of the agglomerates. Preferably, no more than about 10% of the agglomerates are 50% smaller or 50% larger than the mean or target agglomerate size. For example, for an agglomerate of 300 μm, no more than about 10% of the agglomerates will be smaller than about 150 μm or larger than about 450 μm.

A useful method of preparing the agglomerates is described in U.S. Pat. No. 6,503,537, which is incorporated herein. Suitable methods involve mixing preselected amounts of one or more pharmacologically active agent(s) and the micronized, amorphous content containing, dry solid binder in a ratio of between about 100:1 and about 1:500; between about 100:1 and about 1:300 (drug:binder); between about 20:1 to about 1:20 or a ratio of about 1:3 to about 1:10 relative to the amount of the solid binder.

Useful agglomerates may have a strength which ranges from between about 50 mg and about 5,000 mg and most preferably between about 200 mg and about 1,500 mg. The crush strength was tested on a Seiko TMA/SS 120 C Thermomechanical Analyzer available from Seiko Instruments, Inc. Tokyo, Japan, using procedures available from the manufacturer. It should be noted that strength measured in this manner is influenced by the quality and extent of the interparticulate crystalline bonding described herein. However, the size of the agglomerates also plays a role in the measured crush strength. Generally, larger agglomerates require more force to crush than do the smaller particles.

Various pharmaceutical active agents may be utilized. Suitable at least one active pharmaceutical agents include, but are not limited to, an anticholinergic, a corticosteroid, a long acting beta agonist, short acting beta agonist, a phosphodiesterase 4 inhibitor and combinations of two or more thereof. Suitable medicaments may be useful for the prevention or treatment of a respiratory, inflammatory or obstructive airway disease. Examples of such diseases include asthma or chronic obstructive pulmonary disease.

Suitable anticholinergics include (R)-3-[2-hydroxy-2,2-(dithien-2-yl)acetoxy]-1-1[2-(phenypethyl]-1-azoniabicyclo[2.2.2]octane, glycopyrrolate, ipratropium bromide, oxitropium bromide, atropine methyl nitrate, atropine sulfate, ipratropium, belladonna extract, scopolamine, scopolamine methobromide, methscopolamine, homatropine methobromide, hyoscyamine, isopriopramide, orphenadrine, benzalkonium chloride, tiotropium bromide, GSK202405, an individual isomer of any of the above or a pharmaceutically acceptable salt or hydrate of any of the above, or a combination of two or more of the above.

Suitable corticosteroids includes mometasone furoate; beclomethasone dipropionate; budesonide; fluticasone; dexamethasone; flunisolide; triamcinolone; (22R)-6.alpha.,9.alpha.-difluoro-11.beta.,21-dihydroxy-16.alpha.,17.alpha.-propylmethylenedioxy-4-pregnen-3,20-dione, tipredane, GSK685698, GSK799943 or a pharmaceutically acceptable salt or hydrate of any of the above, or a combination of two or more of the above.

Suitable long acting beta agonist include carmoterol, indacaterol, TA-2005, salmeterol, formoterol, or a pharmaceutically acceptable salt or hydrate of any of the above, or a combination of two or more of the above. Suitable short acting beta agonist include albuterol, terbutaline sulfate, bitolterol mesylate, levalbuterol, metaproterenol sulfate, pirbuterol acetate or a pharmaceutically acceptable salt or hydrate of any of the above, or a combination of two or more of the above.

Suitable phosphodiesterase 4 inhibitors include cilomilast, roflumilast, tetomilast, 1-[[5-(1(S)-aminoethyl)-2-[8-methoxy-2-(trifluoromethyl)-5-quinolinyl]-4-oxazolyl]carbonyl]-4(R)-[(cyclopropylcarbonyl)amino]-L-proline, ethyl ester or a pharmaceutically acceptable salt or hydrate of any of the above, or a combination of two or more of the above.

Suitable other APAs include but are not limited to CXCR2 antagonists, muscarinic anatagonists and CXCR3 antagonists.

In certain embodiments of the present invention the at least one active pharmaceutical agent includes a corticosteroid, such as mometasone furoate. Mometasone furoate is an anti-inflammatory corticosteroid having the chemical name, 9,21-Dichloro-11(beta), 17-dihydroxy-16(alpha)-methylpregna-1,4-diene-3,20-dione 17-(2 furoate). It is practically insoluble in water; slightly soluble in methanol, ethanol, and isopropanol; soluble in acetone and chloroform; and freely soluble in tetrahydrofuran. Its partition coefficient between octanol and water is greater than 5000. Mometasone can exist in various hydrated, crystalline and enantiomeric forms, e.g., as a monohydrate.

Several of these compounds could be administered in the form of pharmacologically acceptable esters, salts, solvates, such as hydrates, or solvates of such esters or salts, if any. The term is also meant to cover both racemic mixtures as well as one or more optical isomers. The drug in accordance with the present invention can also be an inhalable protein or a peptide such as insulin, interferons, calcitonins, parathyroid hormones, granulocyte colony-stimulating factor and the like. “Drug” as used herein may refer to a single pharmacologically active entity, or to combinations of any two or more, an example of a useful combination being a dosage form including both a corticosteroid and a β-agonist. A preferred active pharmaceutical agent for use in accordance with the present invention is mometasone furoate.

To be topically effective in the lungs or the upper and/or lower airway passages, it is desirable that the active pharmaceutical agent be delivered as particles of about 10 μm or less. See Task Group on Lung Dynamics, Deposition and Retention Models For Internal Dosimetry of the Human Respiratory Tract, Health Phys., 12, 173, 1966. The ability of a dosage form to actually administer free particles of these therapeutically effectively sized particles is the fine particle fraction. Fine particle fraction is, therefore, a measure of the percentage of bound drug particles released as free particles of drug having a particle size below some threshold during administration. Fine particle fraction can be measured using a multi-stage liquid impinger manufactured by Copley Instruments (Nottingham) LTD using the manufacturer's protocols. In accordance with the present invention, an acceptable fine particle fraction is at least 10% by weight of the drug being made available as free particles having an aerodynamic particle size of 6.8 μm, or less, measured at a flow rate of 60 liters per minute.

The amount of drug administered will vary with a number of factors including, without limitation, the age, sex, weight, condition of the patient, the drug, the course of treatment, the number of doses per day and the like. For mometasone furoate, the amount of drug delivered per dose, i.e. per inhalation, will generally range from about 10.0 μg to about 10,000 μg. Doses of 25 μg, 50 μg, 75 μg, 100 μg, 125 μg, 150 μg, 175 μg, 200 μg, 250 μg, 300 μg, 400 μg and/or 500 μg are preferred.

The solid binder in accordance with the present invention can be any substance which can be provided in, or reduced to, a particle size which is roughly congruent with the size of the particles of the active pharmaceutical agent as previously described. For example, agglomerates of mometasone furoate anhydrous USP will preferably be provided having particles of at least 80% ≦5 μm and at least 95% 10 μm (measured by volume distribution). The solid binder, such as anhydrous lactose, NF will be provided having particles of at least 60% ≦5 μm, at least 90% under 10 μm, and at least 95% ≦20 μm. The average particle size is roughly the same for both and is less than 10 μm.

Suitable solid binders include polyhydroxy aldehydes, polyhydroxy ketones, and amino acids. Preferred polyhydroxy aldehydes and polyhydroxy ketones are hydrated and anhydrous saccharides including, without limitation, lactose, glucose, fructose, galactose, trehalose, sucrose, maltose, raffinose, mannitol, melezitose, starch, xylitol, mannitol, myoinositol, their derivatives, and the like. Particularly useful amino acids include glycine, alanine, betaine and lysine.

Percentages are expressed on a weight basis, unless the context clearly indicates otherwise. The mention of any specific drug substance in this specification or in the claims is intended to encompass not only the base drug, but also pharmaceutically acceptable salts, esters, hydrates and other forms of the drug. Where a particular salt or other form of a drug is mentioned, it is contemplated that other salts or forms can be substituted.

### EXAMPLES

Agglomerates include excipients such as lactose anhydrous, NF (obtained from Kerry Biosciences, Hoffman Estates IL). Magnesium stearate (Peter Greven, Bad Münstereifel, Germany) and colloidal silica (CAB-O-SIL®, Cabot Corporation, Boston, Mass.) are used as the at least one additional functional excipients. The specific surface area of magnesium stearate was 8 m²/g and that of colloidal silica was 200 m²/g. Mometasone furoate (MF) was the APA used in this study.

Micronization of the drug and lactose were performed in-house using a jet-mill (Micron Master, The Jet Pulverizer Co., Inc, Moorestown, N.J.). The average particle size (D_{V50}) of micronized APA (active pharmaceutical agent) and micronized lactose were 1.1 and 2.0 μm, respectively. Particle size measurements of the micronized materials were performed using a HELOS® (Sympatec Inc., Clausthal-Zellerfeld, Germany) laser diffraction system.

Agglomerate Preparation

Drug and excipients were blended in a V-blender (Patterson-Kelley Co., East Stroudsburg, Pa.) equipped with an intensifier bar to provide high shear mixing. APA (or drug) and lactose were blended at APA concentration of about 15% w/w. A typical batch containing only APA and lactose was formulated as a control. When additive was incorporated into the blend, the concentration of lactose was adjusted so that the ratio of drug in the blend was unchanged. Each blend was mixed for about 10 minutes, with the intensifier bar turned on for several minutes. To measure the effect of blending order, magnesium stearate was blended first with either drug or lactose, or last after APA and lactose were blended. When the additive was pre-blended, an additional several minute pre-blending step was performed with the intensifier bar. The blend was formulated into free-flowing agglomerates using the process described in U.S. Pat. No. 6,503,537. The agglomerates were filled into Schering-Plough's TWISTHALER® device that is designed to break agglomerates into particles in the respirable range during an inhalation.

Agglomerate Particle size

The agglomerate particle size distributions were measured by laser diffraction using HELOS® equipped with R6 lens capable of measuring particle sizes between 0.5 and 1770 μm. The GRADIS® (Sympatec Inc., Clausthal-Zellerfeld, Germany) fall shaft is fed by the VIBRI® (Sympatec Inc., Clausthal-Zellerfeld, Germany) feeder that vibrated to the horizontal plane to aid in cascading the agglomerates past the laser.

Aerodynamic Particle Size Distribution

Andersen cascade impaction (ACI) using a glass throat, pre-separator, seven impactor plates and a filter was used to determine the aerodynamic particle size of the agglomerates filled in a Twisthaler® device. Measurements using the first dispensed dose from three inhalers were performed at a 60 L/min flow rate. The drug content contained on each impactor plate (including the casings of the impactor) was assayed using HPLC. Reagents used for ACI and HPLC were methanol, glacial acetic acid and purified water.

Statistical Analysis

All statistical analyses were performed using JMP® software using the Anova (Dunnett post hoc) test, where significance was denoted by p<0.05.

Results and Discussion

Agglomerate Particle Size

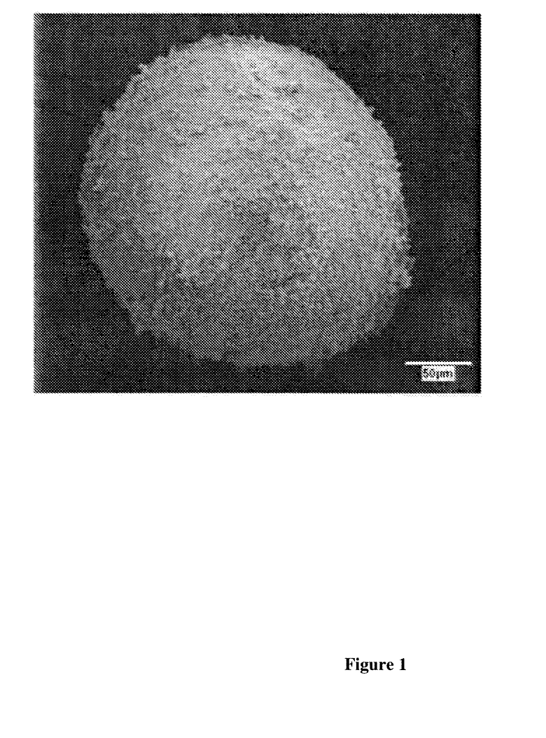

The agglomerate particle size distribution was analyzed by laser diffraction to determine if additives affect the formation and size of the agglomerates. The mean volume diameter of the batch not containing any additional functional excipient was 485.0±23.2 μm. Formulations containing magnesium stearate (MgSt) did not differ significantly in agglomerate particle size compared to the typical batch, where no additive was used (FIG. 1). In addition, the concentration and the order of addition of magnesium stearate was not found to affect agglomerate formation. For all MgSt batches, the range of mean particle size was between 450.8 μm to 512.4 μm. The data demonstrate that the levels of magnesium stearate used in this study did not change the particle size of the bulk agglomerates.

When colloidal silica was pre-blended with lactose, the particle size of the agglomerates decreased (p<0.05) compared to the batch without any additive. The average agglomerate particle size was found to be 384.0 μm compared to 485.0 μm for the typical batch. The specific surface area of colloidal silica (200 m²/g) was an order of magnitude higher than MgSt (8 m²/g).

TABLE 1
Agglomerate particle size distribution of formulations
containing magnesium stearate (MgSt) or colloidal silica (CS)
measured by the Sympatec HELOS ® (mean ± S.D.).

| | Additive | | | |
|---|---|---|---|---|
| | Conc. | | | |
| | (% w/w) | D_{V10} | D_{V50} | D_{V90} |
| No | 0.0 | 330.7 ± 22.4 | 485.1 ± 23.2 | 654.8 ± 31.3 |
| Additive | | | | |
| MgSt | 0.5 | 326.3 ± 49.4 | 476.3 ± 55.7 | 636.8 ± 58.3 |
| blended last | 1.0 | 293.9 ± 2.2 | 465.6 ± 7.8 | 635.6 ± 26.4 |
| MgSt pre-blended | 1.0 | 289.9 ± 24.4 | 468.0 ± 33.0 | 677.3 ± 14.2 |
| with drug | 2.0 | 299.7 ± 32.8 | 457.9 ± 40.4 | 643.5 ± 37.1 |
| MgSt pre-blended | 1.0 | 350.0 ± 49.0 | 512.4 ± 51.8 | 679.3 ± 41.7 |
| with lactose | 2.0 | 280.6 ± 26.4 | 450.8 ± 28.6 | 637.1 ± 42.3 |
| CS pre-blended | 1.0 | 223.3 ± 10.6 | 384.0 ± 12.7 | 577.9 ± 16.8 |
| with lactose | | | | |

Aerodynamic Particle Size

The effects of the concentration of the at least one additional functional excipient and order of addition were studied to determine the effects on the aerodynamic performance of the product. In particular, blending order was investigated to determine if the interparticle forces that modulate the aerodynamic performance are affected by the order of addition. Three different blending sequences were explored: MgSt was blended in the last 3 min of the blending process, pre-blended with drug before it was mixed with lactose, or pre-blended with lactose before the drug was added to it. When colloidal silica was used as the additive, it was pre-blended with lactose before the drug was added to it.

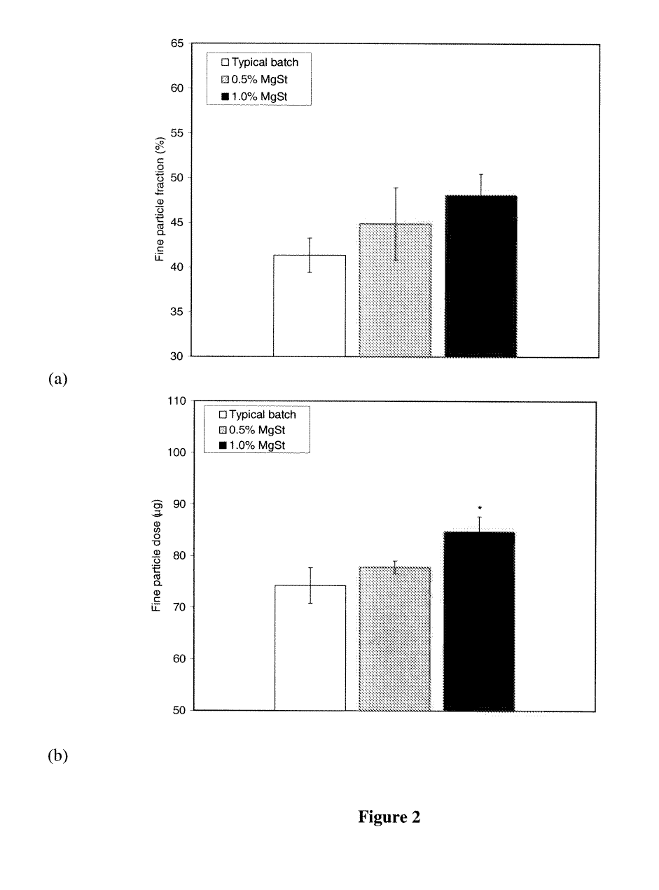

The typical batch not containing any additive yielded a fine particle fraction (FPF, <6.5 μm) of 41.3±1.9% and a fine particle dose (FPD, <6.5 μm) of 74.2±2.7 μg (FIG. 2). When batches containing 0.5% or 1.0% w/w MgSt (added during last 3 min of blending) were analyzed using ACI, both the fine particle fraction and FPD were found to increase proportional to the additive concentration as shown in FIG. 2. The FPD increase was found to be of statistical significance (p<0.05) when 1.0% w/w MgSt was used.

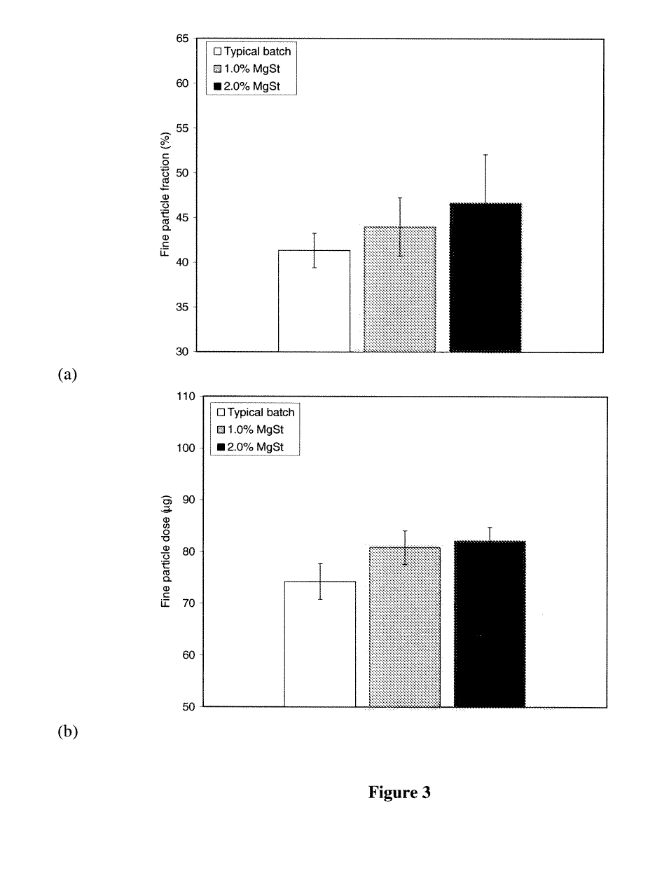

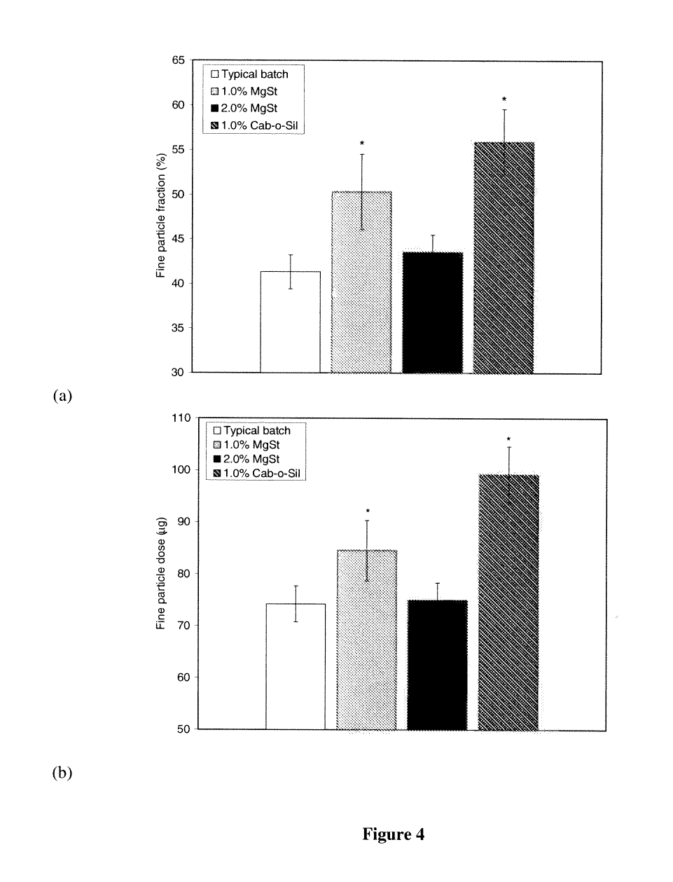

In a different blending sequence, MgSt was pre-mixed with the drug before adding it to the lactose in the V-blender. Two concentrations of MgSt were evaluated −1.0% and 2.0% w/w. An increase in both average fine particle fraction and fine particle dose was observed at both MgSt concentrations when compared to the case with no additive (FIG. 3). An alternate blending sequence where MgSt was pre-blended with lactose before drug was added to it was also explored. Two MgSt concentrations, 1.0% and 2.0% w/w were used. For the 1.0% w/w MgSt case a significant increase (p<0.05) in FPF and FPD were measured compared to the formulation not containing any additive. Interestingly, the formulation containing 2.0% w/w MgSt did not demonstrate a progressive increase in FPD and FPF.

Since 1.0% w/w MgSt pre-blended with lactose resulted in the greatest increase in FPF and FPD among all the cases explore for MgSt, the same blending sequence was used for 1.0% w/w colloidal silica to study its effect on product performance. Colloidal silica imparted the most significant increase in both FPD (99.2±10.5 μg) and FPF (55.9±3.7%) compared to the no additional functional excipient batch.

### CONCLUSIONS

This study demonstrated for the first time that at least one additional functional excipient can be used to modify the fine particle fraction and fine particle dose of an agglomerate-based DPI system. Magnesium stearate did not affect the agglomerate formation process at the concentration levels tested (0.5-2.0% w/w), though addition of colloidal silica yielded smaller agglomerates. Colloidal silica resulted in the greatest increase in fine particle fraction and fine particle dose. Magnesium stearate also demonstrated an increase in fine particle dose, though the concentration and blending sequence affected the magnitude of this change. This study showed for the first time that additives can be used to alter the aerodynamic properties of an agglomerate-based dry powder inhaler formulation.

The foregoing descriptions of various embodiments of the invention are representative of various aspects of the invention, and are not intended to be exhaustive or limiting to the precise forms disclosed. Many modifications and variations may occur to those having skill in the art. It is intended that the scope of the invention shall be fully defined solely by the appended claims.

1. An agglomerate useful for an agglomerate based dry powder inhaler comprising at least one active pharmaceutical agent, at least one binder and at least one additional functional excipient capable of changing the fine particle fraction of the delivered dose of the agglomerate.

2. The agglomerate of claim 1, wherein the at least one additional functional excipient is selected from the group consisting sugars, lubricants, antistatic agents, amino acids, peptides, surfactants, phospholipids and combinations thereof.

3. The agglomerate of claim 1, wherein the at least one additional functional excipient is selected from the group consisting of colloidal silica, magnesium stearate, sucrose stearate, lactose, glucose and mannitol, leucine and combinations thereof.

4. The agglomerate of claim 1, wherein the at least one additional functional excipient is a lubricant.

5. The agglomerate of claim 1, wherein the at least one additional functional excipient is present in an amount from about 0.1 to about 10% of the total weight of the agglomerate.

6. The agglomerate of claim 1, wherein the at least one additional functional excipient is present in an amount from about 0.5 to about 2% of the total weight of the agglomerate.

7. The agglomerate of claim 1, wherein the at least one additional functional excipient is present in an amount of about 1.0% of the total weight of the agglomerate.

8. The agglomerate of claim 1, wherein the at least one additional functional excipient is present in an amount of about 0.5% of the total weight of the agglomerate.

9. The agglomerate of claim 1, wherein the at least one active pharmaceutical agent is selected from the group consisting of an anticholinergic, a corticosteroid, a long acting beta agonist, short acting beta agonist, a phosphodiesterase 4 inhibitor and combinations of two or more thereof.

10. The agglomerate of claim 1, wherein the at least one binder is selected from the group consisting of lactose anhydrous NF, lactose monohydrate and combinations thereof.

11. The agglomerate of claim 1, wherein the at least one binder comprises lactose anhydrous NF.

12. The agglomerate of claim 1, wherein the active pharmaceutical agent emitted dose from a dry powder inhaler has a fine particle fraction of greater than about 50%.

13. The agglomerate of claim 1, wherein at least one active pharmaceutical agent emitted dose from a dry powder inhaler has a fine particle fraction of greater than about 70%.

14. The agglomerate of claim 1 wherein the functional excipiet is magnesium stearate and the binder is lactose.

15. An agglomerate comprising at least one active pharmaceutical agent, lactose and colloidal silica.

16. A method of controlling the fine particle dose of an agglomerate particle based dry powder inhaler comprising an agglomerate formulation comprising at least one active pharmaceutical agent, at least one binder and at least one additional functional excipient capable of changing the fine particle fraction of the delivered dose of the agglomerate.

17. The method of claim 16, wherein the at least one additional functional excipient is selected from the group consisting of magnesium stearate and colloidal silica.

18. The method of claim 16, wherein the at least one additional functional excipient is present in an amount from about 0.1 to about 10% of the total weight of the agglomerate.

19. The method of claim 16, wherein the at least one additional functional excipient is present in an amount of about 1.0% of the total weight of the agglomerate.

20. The method of claim 16, wherein the at least one additional functional excipient is present in an amount of about 0.5% of the total weight of the agglomerate.
